(19) United States
(12) Patent Application Publication
(10) Pub. No.: US 2026/0109933 A1
(43) Pub. Date: Apr. 23, 2026

(54) ONLINE ENRICHMENT AND AUTOMATIC SORTING APPARATUS AND METHOD FOR DEEP-SEA MICROORGANISMS UNDER HIGH-PRESSURE ENVIRONMENTS

(71) Applicant: GUANGDONG LABORATORY OF SOUTHERN OCEAN SCIENCE AND ENGINEERING (GUANGZHOU), Guangdong (CN)

(72) Inventors: Jingchun FENG, Guangdong (CN); Si ZHANG, Guangdong (CN); Jianzhen LIANG, Guangdong (CN); Zhifeng YANG, Guangdong (CN)

(73) Assignee: GUANGDONG LABORATORY OF SOUTHERN OCEAN SCIENCE AND ENGINEERING (GUANGZHOU), Guangdong (CN)

(21) Appl. No.: 19/365,229

(22) Filed: Oct. 22, 2025

(30) Foreign Application Priority Data
Oct. 22, 2024 (CN) 202411477979.1

(51) Int. Cl. C12M 1/34; C12M 1/00; C12M 1/22; C12M 1/26; C12M 1/36

(52) U.S. Cl. CPC C12M 41/40; C12M 23/10; C12M 23/58; C12M 29/06; C12M 33/14; C12M 41/12; C12M 41/44; C12M 41/48; C12M 47/02

(57) ABSTRACT

The present invention provides an online enrichment and automatic sorting apparatus and method for deep-sea microorganisms under high-pressure environments. High-pressure and specific temperature environments of deep seas are simulated to achieve multi-level enrichment. Based on changes in environmental indicators observed online within the enrichment apparatus, an optimal time point for microbial subculture is automatically determined, and a culture is transferred to a next-level enrichment apparatus, thereby effectively addressing problems such as difficulty in transferring enriched microorganisms at the optimal time point under the high-pressure environments and complex manual operations, and improving the preliminary enrichment efficiency for obtaining individual species of deep-sea microorganisms. Furthermore, in the present invention, automatic streaking separation and automatic secondary sorting of enriched materials, and automatic transfer of specific individual colonies to pure culture kettles are performed on the culture medium under high-pressure environments.

### CROSS-REFERENCE TO RELATED APPLICATION

This application claims the priority benefit of China Application No. 202411477979.1, filed on Oct. 22, 2024. The entirety of the above-mentioned patent application is hereby incorporated by reference herein and made a part of this specification.

### TECHNICAL FIELD

The present invention relates to the technical field of marine microorganism enrichment culture and individual microorganism sorting, and more specifically, to an online enrichment and automatic sorting apparatus and method for deep-sea microorganisms under high-pressure environments.

### BACKGROUND

Deep-sea ecosystem has given rise to various special habitats such as cold seeps and hydrothermal vents due to its unique environmental conditions. Under extreme environments such as high pressure and low/high temperature, deep-sea microorganisms possess remarkable uniqueness and diversity in gene structure and physiological metabolism; these deep-sea microorganisms play an irreplaceable role in promoting marine energy transfer and material cycling, as well as maintaining the stability of marine and even global ecosystems.

To investigate the unique functions of a particular deep-sea microorganism within the marine ecosystem, it is necessary to sort and purely cultivate individual organisms, thereby determining their microbial characteristics, such as physiological features of growth and metabolism. However, current technologies for sorting and pure cultivation of deep-sea microorganisms remain immature. After deep-sea microorganisms are sampled and transferred to the laboratory, they are removed from their in situ high-pressure environment, making them prone to cell rupture and difficult to survive, which renders efficient isolation and cultivation of individual microbial species exceptionally challenging. Therefore, enrichment and pure cultivation of microorganisms using a high-pressure culture apparatus is an indispensable approach in the research field of deep-sea microorganisms.

For the enrichment of deep-sea microorganisms under simulated environmental conditions, an enrichment and multi-level purification apparatus and method for deep-sea microorganisms under high-pressure environments are disclosed in the prior art. In existing high-pressure enrichment technologies, the enriched materials are mainly sampled by a sampler for instrument detection and analysis. However, the reactant status of the enriched microbial liquid inside the apparatus cannot be observed online, and the enriched materials are not transferred based on the reactant status, making it difficult to conduct microbial subculturing at the optimal time point of metabolite changes. This results in limitations such as complex manual operations and low microorganism sorting activity.

For the sorting of deep-sea microorganisms under simulated environmental conditions, a system and method for high-throughput automatic screening of individual deep-sea bacterial colonies under temperature and pressure retention conditions are disclosed in the prior art. In existing automatic sorting technologies, the enriched microbial liquid is separated by spraying, which leads to low biological separation and overlapping colonies, hindering rapid growth and effective separation of individual microbial colonies. In addition, a biological coating device is further disclosed in the prior art; in the automatic sorting technology, the microbial liquid is coated in a culture medium through the mechanical arm for separation. However, when applied under high-pressure environments, the culture medium in a petri dish may be uneven or tilted. This potentially causes a coating rod tip to fall off, resulting in failed streaking and difficulty in recovering a pipette tip for re-coating. As a result, there are challenges such as complex manual sorting operations and difficulty in automatic secondary sorting after automatic coating failure.

In summary, problems such as complex manual operations in microorganism enrichment, low separation degree of enriched materials, low activity in microorganism sorting, and difficulty in automatic secondary sorting hinder the efficient acquisition of individual species of deep-sea microorganisms.

### SUMMARY

To overcome the shortcomings of the complex manual operations in deep-sea microorganism enrichment, low separation of enriched materials, low activity in microorganism sorting, and difficulty in automatic secondary sorting, the present invention provides an online enrichment and automatic sorting apparatus and method for deep-sea microorganisms under high-pressure environments. Through online monitoring of enrichment environment, automatic determination of an optimal time for microbial subculturing, automatic streaking separation of enriched materials, automatic secondary sorting, and automatic transfer of specific individual colonies, cultivability and acquisition efficiency of individual species of deep-sea microorganisms are improved. This provides an essential foundation for advancing the understanding of deep-sea life sciences and enhancing the efficiency of biological resource development and application.

To address the above technical problems, the technical solutions of the present invention are as follows:

An online enrichment apparatus for deep-sea microorganisms under high-pressure environments is provided and includes: a multi-level enrichment unit, a first pressure control unit, a first temperature control unit, a first data acquisition unit, and a first central control unit.

The multi-level enrichment unit includes a liquid injection mechanism, a liquid transfer mechanism, and at least two microorganism culture kettles connected in series; the liquid injection mechanism is used to inject a microbial culture solution into each microorganism culture kettle; a primary microorganism culture kettle contains in situ sediment samples for microorganism enrichment; and the liquid transfer mechanism is used to transfer a microbial liquid from a previous-level microorganism culture kettle to a next-level microorganism culture kettle.

The first pressure control unit and the first temperature control unit each are connected to the first central control unit and each microorganism culture kettle respectively, and are respectively used to control pressure and temperature within each microorganism culture kettle.

The first data acquisition unit is connected to the first central control unit, and the first data acquisition unit is used to acquire environmental data from each microorganism culture kettle in real time and transmit the environmental data acquired to the first central control unit in real time.

The first central control unit is further connected to each microorganism culture kettle, the liquid injection mechanism, the liquid transfer mechanism, the first pressure control unit, and the first temperature control unit respectively; and the first central control unit is used to control the online enrichment apparatus for deep-sea microorganisms under high-pressure environments according to the data acquired by the first data acquisition unit.

Preferably, in the multi-level enrichment unit, the liquid injection mechanism includes a continuous liquid injection pump and a plurality of liquid injection valves, each liquid injection valve is correspondingly provided on a microbial culture solution inlet pipeline of each microorganism culture kettle, and the continuous liquid injection pump is connected to each liquid injection valve.

The liquid transfer mechanism includes a sample transfer injection pump, multiple sets of filters and sample transfer valves, as well as a plunger pump; the sample transfer injection pump is respectively connected to all microorganism culture kettles except for a last-level microorganism culture kettle, and one set of filters and the sample transfer valve is provided on a connection pipeline between each two microorganism culture kettles connected in series; and the plunger pump is connected to the last-level microorganism culture kettle.

Each microorganism culture kettle has a same structure, with a magnetic stirrer and a sampling valve provided at a bottom; and
tops of all the microorganism culture kettles except for a last-level microorganism culture kettle are each provided with a sample transfer piston, and middle parts of all the microorganism culture kettles are each provided with protruding circular rings; the sample transfer piston is used to reduce a pressure difference required for transferring the microbial liquid from the previous-level microorganism culture kettle to the next-level microorganism culture kettle; and the protruding circular ring is used to prevent the sample transfer piston from being excessively pressurized.

Preferably, the first pressure control unit includes: a gas pressurization system, a plurality of gas injection valves, and a plurality of PID control valves.

Each microorganism culture kettle is provided with one gas injection valve; the gas pressurization system is connected to each gas injection valve via pipelines, and the gas pressurization system is connected to the first central control unit, and is used to perform gas pressurization on each microorganism culture kettle.

Each microorganism culture kettle is further provided with one PID control valve, and each PID control valve is connected to the central control unit and is used to control formation of a continuous pressure environment within the microorganism culture kettles connected in series.

The first temperature control unit includes: an air conditioning system.

The air conditioning system is respectively connected to the first central control unit and each microorganism culture kettle, and is used to control the temperature within each microorganism culture kettle.

The first data acquisition unit includes: a pressure sensing system, a temperature sensing system, a Raman measurement system, a dissolved oxygen measurement system, and a pH measurement system.

The pressure sensing system and the temperature sensing system are each connected to the first central control unit and each microorganism culture kettle respectively, and are respectively used to acquire the pressure and temperature within each microorganism culture kettle in real time and transmit the pressure and temperature acquired to the first central control unit in real time.

The Raman measurement system, the dissolved oxygen measurement system, and the pH measurement system are each connected to the first central control unit and each microorganism culture kettle respectively, and are respectively used to perform real-time online observing of changes in chemical substance content, dissolved oxygen content, and pH value within each microorganism culture kettle, and transmit the changes in the chemical substance content, the dissolved oxygen content, and the pH value within each microorganism culture kettle that are observed to the first central control unit in real time.

The present invention further provides an online automatic sorting apparatus for deep-sea microorganisms under high-pressure environments, and the apparatus includes: an automatic sorting unit, a second pressure control unit, a second temperature control unit, a second data acquisition unit, and a second central control unit.

The automatic sorting unit includes a sorting operation kettle and a plurality of pure culture kettles; and the sorting operation kettle is connected to each pure culture kettle.

The sorting operation kettle includes an internal three-axis moving mechanism and at least one streaking pen tip box, where a plurality of streaking pen tips are placed inside the streaking pen tip box, a moving Z-axis of the three-axis moving mechanism is provided with a mechanical arm capable of picking up the streaking pen tips from the streaking pen tip box, and the mechanical arm is used to drive the streaking pen tips to perform sorting operations; and X-axis and Y-axis planes of the three-axis moving mechanism are provided with a culture area composed of a plurality of petri dishes.

The second pressure control unit and the second temperature control unit are each connected to the second central control unit respectively, the sorting operation kettle, and each pure culture kettle, and are respectively used to control pressure and temperature within the sorting operation kettle, and each pure culture kettle.

The second data acquisition unit is connected to the second central control unit and is used to acquire image data of the culture area inside the sorting operation kettle in real time and transmit the image data acquired to the second central control unit in real time.

The second central control unit is further connected to the mechanical arm, the second pressure control unit, and the second temperature control unit, where the second central control unit is used to control the online automatic sorting apparatus for deep-sea microorganisms under high-pressure environments according to the image data acquired by the second data acquisition unit.

Preferably, in the automatic sorting unit, the sorting operation kettle includes an upper kettle body and a lower kettle body that are hermetically connected.

A pressure-resistant viewing window is provided on an upper surface of the upper kettle body for observing an interior of the sorting operation kettle.

A microbial liquid reservoir, the three-axis moving mechanism, at least one streaking pen tip box, the plurality of petri dishes, a pen tip recovery reservoir, and a sorting transfer port are provided in the lower kettle body.

The microbial liquid reservoir is used to hold a to-be-sorted microbial liquid.

The three-axis moving mechanism is provided with three X-axis, Y-axis, and Z-axis sliding rails that vertically intersect, where the mechanical arm is provided on the Z-axis sliding rail.

The mechanical arm includes: a vertical rod, and a magnetic force sensor and a fixing block provided at both ends of the vertical rod; and the magnetic force sensor is respectively connected to the second central control unit and the fixing block, the magnetic force sensor provides a magnetic force to the fixing block, and the fixing block picks up the streaking pen tip by the magnetic force.

The mechanical arm is used to perform the following operations: picking up the streaking pen tip from the streaking pen tip box and installing the streaking pen tip in the streaking pen tip box; driving the streaking pen tip to dip the microbial liquid in the microbial liquid reservoir and the petri dishes; driving the streaking pen tip to perform streaking and sorting in the petri dishes; and releasing the streaking pen tip in the pen tip recovery reservoir and the sorting transfer port.

A special culture medium for cultivating special individual colonies is pre-filled in the petri dishes to sort required special microbial species.

The pen tip recovery reservoir is used to collect used streaking pen tips.

The sorting transfer port is connected to each pure culture kettle via pipelines and quick-connect interfaces, where the sorting transfer port is used to transfer the streaking pen tip carrying individual colonies after sorting to the pure culture kettle.

Preferably, the second pressure control unit includes: a gas pressurization system, an exhaust valve, and a plurality of gas injection valves.

The sorting operation kettle and each pure culture kettle are respectively provided with one gas injection valve; and the gas pressurization system is connected to each gas injection valve via pipelines, and the gas pressurization system is connected to the second central control unit and used to perform gas pressurization for the sorting operation kettle and each pure culture kettle.

The exhaust valve is provided on the sorting operation kettle and is used to exhaust gas from the sorting operation kettle.

The second temperature control unit includes: an air conditioning system.

The air conditioning system is respectively connected to the second central control unit, the sorting operation kettle, and each pure culture kettle, and is used to control the temperature within the sorting operation kettle, and each pure culture kettle.

The second data acquisition unit includes a pressure sensing system, a temperature sensing system, and an imaging system;

The pressure sensing system and the temperature sensing system are each connected to the second central control unit, the sorting operation kettle, and each pure culture kettle respectively, and are used to acquire the pressure and temperature within the sorting operation kettle, and each pure culture kettle in real time and transmit the pressure and temperature acquired to the second central control unit in real time.

The imaging system is provided at a top of the sorting operation kettle and is connected to the second central control unit, where the imaging system is used to acquire image data of the culture area inside the sorting operation kettle in real time and transmit the image data acquired to the second central control unit in real time.

The present invention further provides an online enrichment and automatic sorting apparatus for deep-sea microorganisms under high-pressure environments, and the apparatus includes an enrichment apparatus and a sorting apparatus connected in sequence, where the enrichment apparatus is specifically the online enrichment apparatus for deep-sea microorganisms under high-pressure environments, and the sorting apparatus is specifically the online automatic sorting apparatus for deep-sea microorganisms under high-pressure environments.

Preferably, a last-level microorganism culture kettle in the multi-level enrichment unit is connected to the microbial liquid reservoir inside the sorting operation kettle; and
the microbial liquid reservoir is used to hold the to-be-sorted microbial liquid enriched by the last-level microorganism culture kettle.

The present invention further provides an online enrichment and automatic sorting method for deep-sea microorganisms under high-pressure environments based on the online enrichment and automatic sorting apparatus for deep-sea microorganisms under high-pressure environments, and the method includes the following steps:
S1: cleaning and sterilizing each microorganism culture kettle, a sorting operation kettle, and each pure culture kettle, and testing pressure leak of each microorganism culture kettle, the sorting operation kettle, and each pure culture kettle; and installing each unit of the apparatus and performing initialization configuration;
S2: transferring sampled in situ sediment samples to a primary microorganism culture kettle to initiate microorganism enrichment;
S3: using a first data acquisition unit to acquire environmental data of each microorganism culture kettle in real time and transmit the environmental data acquired to a first central control unit in real time; where,
started from the primary microorganism culture kettle, when the environmental data in a previous-level microorganism culture kettle meets a first preset condition, the first central control unit controls a liquid transfer mechanism to transfer a microbial liquid from the previous-level microorganism culture kettle to a next-level microorganism culture kettle for further enrichment culture;
S4: when the environmental data in a last-level microorganism culture kettle meets a second preset condition, completing an enrichment operation, and transferring a microbial liquid from the last-level microorganism culture kettle to the sorting operation kettle;
S5: using a second data acquisition unit to acquire image data of a culture area inside the sorting operation kettle in real time and transmit the image data acquired to a second central control unit in real time; and controlling the mechanical arm by the second central control unit to perform a plurality of streaking operations in the culture area for sorting and culture for a preset duration; and
S6: when the second central control unit identifies the growth of multiple individual colonies in a petri dish from the image data of the culture area, controlling the mechanical arm by the second central control unit to transfer each individual colony to each pure culture kettle, and completing the sorting operation.

Preferably, in step S1, the initialization configuration includes: using a liquid injection mechanism to inject a pre-prepared microbial culture solution into each microorganism culture kettle at a specified flow rate; pre-preparing a special culture medium for each petri dish in the sorting operation kettle, and pre-injecting the pre-prepared special culture solution into each pure culture kettle; using a first pressure control unit and a second pressure control unit to perform gas pressurization on each microorganism culture kettle, the sorting operation kettle, and each pure culture kettle to simulate a deep-sea high-pressure environment; and using a first temperature control unit and a second temperature control unit to control the temperature of each microorganism culture kettle, the sorting operation kettle, and each pure culture kettle to maintain the special deep-sea temperature environment.

In step S5, each streaking operation includes:
controlling, by the second central control unit, the mechanical arm to move to a streaking pen tip box and to picks up the streaking pen tip, and subsequently controlling, by the second central control unit, the mechanical arm to move to the petri dish for streaking; after streaking, controlling, by the second central control unit, the mechanical arm to depart from the culture area, move to a pen tip recovery reservoir, and release the streaking pen tip.

In step S6, the controlling the mechanical arm by the second central control unit to transfer each individual colony to each pure culture kettle includes:
controlling, by the second central control unit, the mechanical arm to pick up the streaking pen tip, move to an individual colony for carrying, then be transferred to a sorting transfer port and release the streaking pen tip; and transferring the streaking pen tip carrying the individual colony to the pure culture kettle.

Compared with the prior art, the beneficial effects of the technical solutions of the present invention are as follows:

The present invention provides an online enrichment apparatus for deep-sea microorganisms under high-pressure environments. High-pressure and specific temperature environments of deep seas are simulated to achieve multi-level enrichment. Based on changes in environmental indicators observed online within the enrichment apparatus, an optimal time point for microbial subculture is automatically determined, and a culture is transferred to a next-level enrichment apparatus, thereby effectively addressing problems such as difficulty in transferring enriched microorganisms at the optimal time point under the high-pressure environments and complex manual operations, and improving the preliminary enrichment efficiency for obtaining individual species of deep-sea microorganisms.

In addition, the present invention further provides an online automatic sorting apparatus for deep-sea microorganisms under high-pressure environments. Through the apparatus, automatic streaking separation and automatic secondary sorting of enriched material, and automatic transfer of specific individual colonies to pure culture kettles are performed on the culture medium under high-pressure environments. This addresses the problems of overlapping individual colonies, low separation degree, complex manual operations, and difficulty in automatic secondary sorting under the high-pressure environments in existing high-pressure sorting technologies, thereby greatly enhancing the sorting efficiency of the deep-sea microorganisms under the high-pressure environments.

In the present invention, the enrichment technology and the sorting technology are combined to form an online enrichment and automatic sorting apparatus. The apparatus can perform continuous multi-level enrichment on the cultures under high-pressure environments through dilution subculturing, and monitor environmental indicators of the culture system online. This achieves integration of enrichment and sorting apparatuses, transfer of the cultures under constant pressure and aseptic conditions, automatic streaking and sorting of the culture medium within the apparatus, and online monitoring of growth status of individual colonies, thereby improving the cultivability and acquisition efficiency of individual species of deep-sea microorganisms and providing an essential foundation for advancing the understanding of deep-sea life sciences and enhancing the efficiency of biological resource development and application.

### BRIEF DESCRIPTION OF DRAWINGS

FIG. 1 is a framework diagram of an online enrichment apparatus for deep-sea microorganisms under high-pressure environments provided in Embodiment 1.

FIG. 2 is a framework diagram of an online automatic sorting apparatus for deep-sea microorganisms under high-pressure environments provided in Embodiment 2.

FIG. 3 is a framework diagram of an online enrichment and automatic sorting apparatus for deep-sea microorganisms under high-pressure environments provided in Embodiment 3.

FIG. 4 is a mechanical structure diagram of an online enrichment and automatic sorting apparatus for deep-sea microorganisms under high-pressure environments provided in Embodiment 4.

FIG. 5 is a top view of an interior of a sorting operation kettle provided in Embodiment 4.

FIG. 6 is a schematic diagram of connection between a central control unit and other components provided in Embodiment 4.

FIG. 7 is a flow chart of an online enrichment and automatic sorting method for deep-sea microorganisms under high-pressure environments provided in Embodiment 5.

FIG. 8 is a flow chart of specific operation for online enrichment and automatic sorting provided in Embodiment 5.

### DETAILED DESCRIPTION OF EMBODIMENTS

The drawings are for exemplary illustration only and shall not be construed as limitations to this patent.

To better illustrate this embodiment, certain components in the drawings may be omitted, enlarged, or reduced, and do not represent the dimensions of the actual product.

For those skilled in the art, it is understandable that certain well-known structures and descriptions thereof in the drawings may be omitted.

The technical solutions of the present invention are further illustrated below in conjunction with the drawings and embodiments.

### Embodiment 1

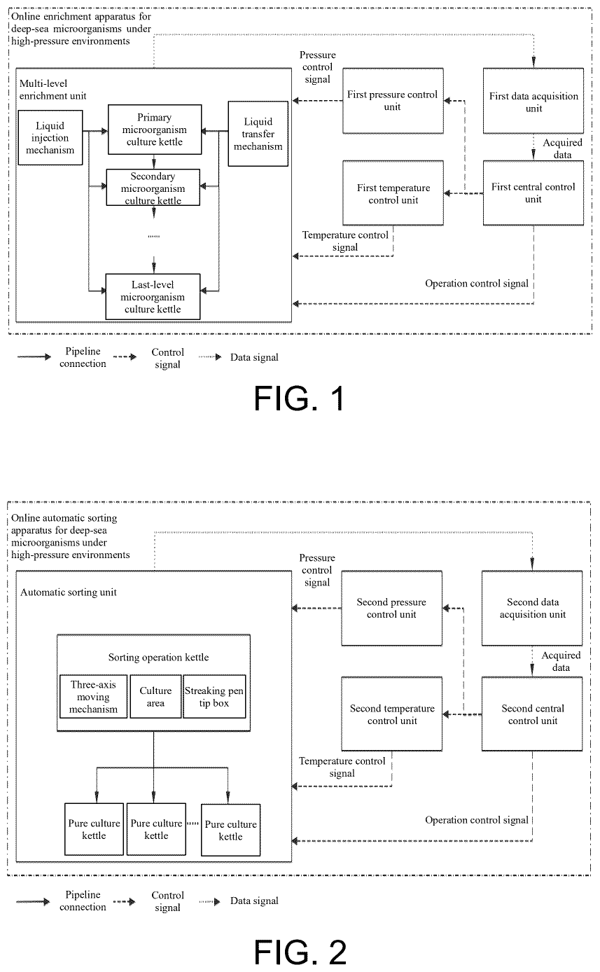

As shown in FIG. 1, this embodiment provides an online enrichment apparatus for deep-sea microorganisms under high-pressure environments, and the apparatus includes: a multi-level enrichment unit, a first pressure control unit, a first temperature control unit, a first data acquisition unit, and a first central control unit.

The multi-level enrichment unit includes a liquid injection mechanism, a liquid transfer mechanism, and at least two microorganism culture kettles connected in series; the liquid injection mechanism is used to inject a microbial culture solution into each microorganism culture kettle; a primary microorganism culture kettle contains in situ sediment samples for microorganism enrichment; and the liquid transfer mechanism is used to transfer a microbial liquid from a previous-level microorganism culture kettle to a next-level microorganism culture kettle.

The first pressure control unit and the first temperature control unit are each connected to the first central control unit and each microorganism culture kettle respectively, and are respectively used to control pressure and temperature within each microorganism culture kettle.

The first data acquisition unit is connected to the first central control unit, and the first data acquisition unit is used to acquire environmental data from each microorganism culture kettle in real time and transmit the environmental data acquired to the first central control unit in real time.

The first central control unit is further respectively connected to each microorganism culture kettle, the liquid injection mechanism, the liquid transfer mechanism, the first pressure control unit, and the first temperature control unit; and the first central control unit is used to control the online enrichment apparatus for deep-sea microorganisms under high-pressure environments according to the data acquired by the first data acquisition unit.

In a specific implementation process, prior to deep-sea microorganism enrichment, each microorganism culture kettle is cleaned and sterilized, and for pressure leak of each microorganism culture kettle is tested; subsequently, each unit of the entire apparatus is installed, all sensors and other electronic instruments within the apparatus are connected to the first central control unit, and the instruments are activated and connected to software of the first central control unit;
sampled in situ sediment samples are transferred to a primary microorganism culture kettle to initiate microorganism enrichment;
a first data acquisition unit is used to acquire environmental data of each microorganism culture kettle in real time and transmit the environmental data acquired to a first central control unit in real time;
started from the primary microorganism culture kettle, when the environmental data in a previous-level microorganism culture kettle meets a first preset condition, the first central control unit controls a liquid transfer mechanism to transfer a microbial liquid from the previous-level microorganism culture kettle to a next-level microorganism culture kettle for further enrichment culture; and
when the environmental data in a last-level microorganism culture kettle meets a second preset condition, a multi-level enrichment operation is completed.

By simulating high-pressure and specific temperature environments of deep seas, the apparatus automatically determines an optimal time point for microbial subculture and transfers a culture to a next-level enrichment apparatus based on changes in environmental indicators observed online within the enrichment apparatus, thereby effectively addressing problems such as difficulty in transferring enriched microorganisms at the optimal time point under the high-pressure environments and complex manual operations, and improving the preliminary enrichment efficiency for obtaining individual species of deep-sea microorganisms.

### Embodiment 2

As shown in FIG. 2, this embodiment provides an online automatic sorting apparatus for deep-sea microorganisms under high-pressure environments, and the apparatus includes: an automatic sorting unit, a second pressure control unit, a second temperature control unit, a second data acquisition unit, and a second central control unit.

The automatic sorting unit includes a sorting operation kettle and a plurality of pure culture kettles; and the sorting operation kettle is connected to each pure culture kettle.

The sorting operation kettle includes an internal three-axis moving mechanism and at least one streaking pen tip box, where a plurality of streaking pen tips are placed inside the streaking pen tip box, a moving Z-axis of the three-axis moving mechanism is provided with a mechanical arm capable of picking up the streaking pen tips from the streaking pen tip box, and the mechanical arm is used to drive the streaking pen tips to perform sorting operations; and X-axis and Y-axis planes of the three-axis moving mechanism are provided with a culture area composed of a plurality of petri dishes.

The second pressure control unit and the second temperature control unit are respectively connected to the second central control unit, the sorting operation kettle, and each pure culture kettle, and are respectively used to control pressure and temperature within the sorting operation kettle, and each pure culture kettle.

The second data acquisition unit is connected to the second central control unit and is used to acquire image data of the culture area inside the sorting operation kettle in real time and transmit the image data acquired to the second central control unit in real time.

The second central control unit is further connected to the mechanical arm, the second pressure control unit, and the second temperature control unit, where the second central control unit is used to control the online automatic sorting apparatus for deep-sea microorganisms under high-pressure environments according to the image data acquired by the second data acquisition unit.

In a specific implementation process, prior to deep-sea microorganism sorting, the sorting operation kettle and each pure culture kettle are cleaned and sterilized, and pressure leak of the sorting operation kettle and each pure culture kettle is tested; subsequently, each unit of the entire apparatus is installed, and all sensors and other electronic instruments within the apparatus are connected to the central control unit, and the instruments are activated, and connected to software of the second central control unit.

The to-be-sorted microbial liquid is transferred to the sorting operation kettle.

A second data acquisition unit is used to acquire image data of a culture area inside the sorting operation kettle in real time and transmit the image data acquired to a second central control unit in real time; and the second central control unit controls the mechanical arm to perform a plurality of streaking operations in the culture area for sorting and culture for a preset duration.

When the second central control unit identifies the growth of multiple individual colonies in a petri dish according to the image data of the culture area, the second central control unit controls the mechanical arm to transfer each individual colony to each pure culture kettle, and the sorting operation is completed.

Through the apparatus, automatic streaking separation and automatic secondary sorting of enriched materials, and automatic transfer of specific individual colonies to pure culture kettles are performed on the culture medium under high-pressure environments. This addresses the problems of overlapping individual colonies, low separation degree, complex manual operations, and difficulty in automatic secondary sorting under the high-pressure environments in existing high-pressure sorting technologies, thereby greatly enhancing the sorting efficiency of the deep-sea microorganisms under the high-pressure environments.

### Embodiment 3

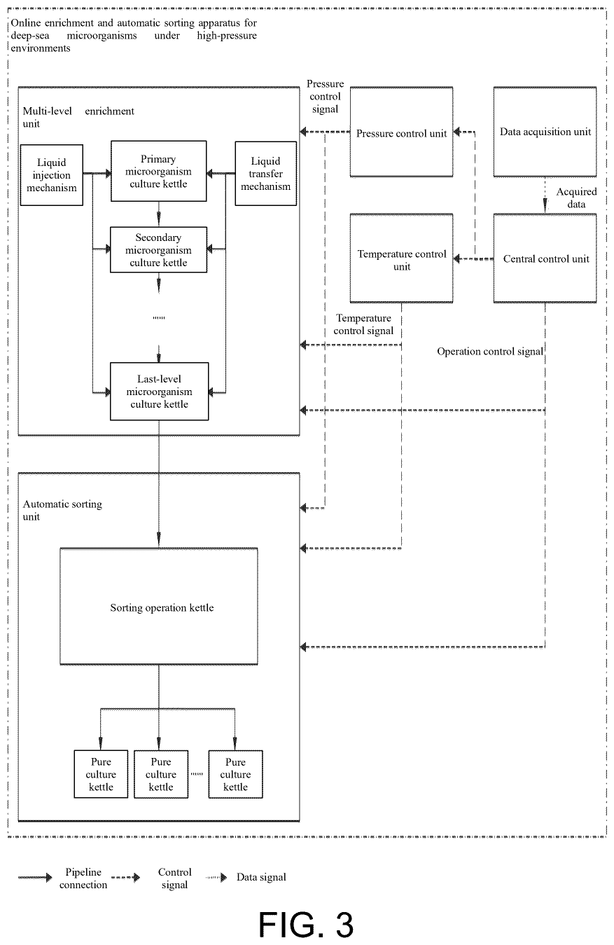

As shown in FIG. 3, this embodiment provides an online enrichment and automatic sorting apparatus for deep-sea microorganisms under high-pressure environments based on the online enrichment apparatus for deep-sea microorganisms under high-pressure environments described in Embodiment 1 and the online automatic sorting apparatus for deep-sea microorganisms under high-pressure environments described in Embodiment 2, and the apparatus includes: a multi-level enrichment unit, an automatic sorting unit, a pressure control unit, a temperature control unit, a data acquisition unit, and a central control unit.

In this embodiment, the pressure control unit, the temperature control unit, the data acquisition unit, and the central control unit are each integrated with the functions of the first pressure control unit, the second pressure control unit, the first temperature control unit, the second temperature control unit, the first data acquisition unit, and the second data acquisition unit, the first central control unit and the second central control unit, and are respectively used to control the pressure, temperature, data acquisition, and specific operations of the entire apparatus (including the enrichment and sorting apparatuses).

The multi-level enrichment unit includes a liquid injection mechanism, a liquid transfer mechanism, and at least two microorganism culture kettles connected in series; the liquid injection mechanism is used to inject a microbial culture solution into each microorganism culture kettle; a primary microorganism culture kettle contains in situ sediment samples for microorganism enrichment; and the liquid transfer mechanism is used to transfer a microbial liquid from a previous-level microorganism culture kettle to a next-level microorganism culture kettle.

The automatic sorting unit includes a sorting operation kettle and a plurality of pure culture kettles; and the sorting operation kettle is connected to each pure culture kettle.

The sorting operation kettle includes an internal three-axis moving mechanism and at least one streaking pen tip box, where a plurality of streaking pen tips are placed inside the streaking pen tip box, a moving Z-axis of the three-axis moving mechanism is provided with a mechanical arm capable of picking up the streaking pen tips from the streaking pen tip box, and the mechanical arm is used to drive the streaking pen tips to perform sorting operations; and X-axis and Y-axis planes of the three-axis moving mechanism are provided with a culture area composed of a plurality of petri dishes.

A last-level microorganism culture kettle in the multi-level enrichment unit is connected to the sorting operation kettle.

The pressure control unit and the temperature control unit are respectively connected to the central control unit, the sorting operation kettle, each microorganism culture kettle, and each pure culture kettle, and are used to control pressure and temperature within the sorting operation kettle, each microorganism culture kettle.

The data acquisition unit is connected to the central control unit and is used to acquire environmental data from each microorganism culture kettle and image data from the culture area inside the sorting operation kettle in real time, and to transmit the acquired data to the central control unit in real time.

The central control unit is further connected to each microorganism culture kettle, the liquid injection mechanism, the liquid transfer mechanism, the mechanical arm, the pressure control unit, and the temperature control unit respectively; and the central control unit is used to control the online enrichment and automatic sorting apparatus for deep-sea microorganisms according to the data acquired by the data acquisition unit.

In a specific implementation process, prior to deep-sea microorganism enrichment, each microorganism culture kettle, the sorting operation kettle and each pure culture kettle are cleaned and sterilized, and pressure leak of each microorganism culture kettle, the sorting operation kettle and each pure culture kettle is tested; subsequently, each unit of the entire apparatus is installed, and all sensors and other electronic instruments within the apparatus are connected to the central control unit, and the instruments are activated, and connected to software of the second central control unit;
sampled in situ sediment samples are transferred to a primary microorganism culture kettle to initiate microorganism enrichment;

A data acquisition unit is used to acquire environmental data of each microorganism culture kettle in real time and transmit the environmental data acquired to the central control unit in real time.

Started from the primary microorganism culture kettle, when the environmental data in a previous-level microorganism culture kettle meets a first preset condition, the central control unit controls a liquid transfer mechanism to transfer a microbial liquid from the previous-level microorganism culture kettle to a next-level microorganism culture kettle for further enrichment culture; and
When the environmental data in a last-level microorganism culture kettle meets a second preset condition, an enrichment operation is completed, and a microbial liquid from the last-level microorganism culture kettle is transferred to the sorting operation kettle.

Then, the data acquisition unit is used to acquire image data from the culture area inside the sorting operation kettle in real time and transmit the acquired image data to the central control unit in real time.

The central control unit controls the mechanical arm to continuously pick up and release the streaking pen tip, and drives the streaking pen tip to perform streaking operations in each petri dish in the culture area for sorting and culture for a preset duration.

When the central control unit identifies the growth of multiple individual colonies in a petri dish according to the image data of the culture area, the central control unit controls the mechanical arm to transfer each individual colony to each pure culture kettle, and the sorting operation is completed.

The apparatus can perform continuous multi-level enrichment on cultures under high-pressure environments through dilution subculturing, and monitor environmental indicators of the culture system online. This achieves integration of enrichment and sorting apparatuses, transfer of the cultures under constant pressure and aseptic conditions, automatic streaking and sorting of the culture medium within the apparatus, and online monitoring of growth status of individual colonies, thereby improving the cultivability and acquisition efficiency of individual species of deep-sea microorganisms.

### Embodiment 4

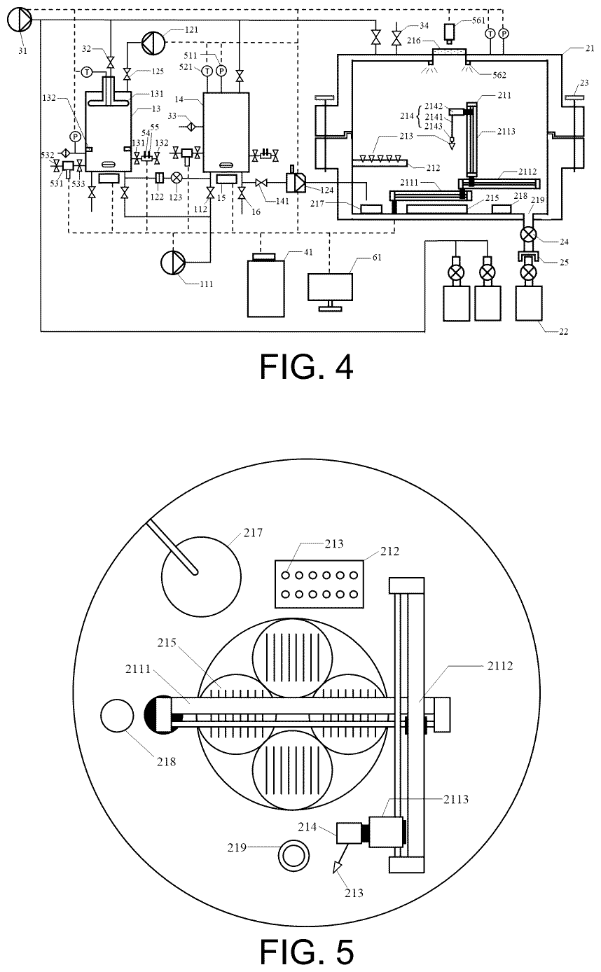

As shown in FIG. 4, based on the online enrichment and automatic sorting apparatus for deep-sea microorganisms under high-pressure environments described in Embodiment 3, this embodiment provides an online enrichment and automatic sorting apparatus for deep-sea microorganisms under high-pressure environments, and the apparatus includes: a multi-level enrichment unit 1, an automatic sorting unit 2, a pressure control unit 3, a temperature control unit 4, a data acquisition unit 5, and a central control unit 6.

The multi-level enrichment unit 1 includes a liquid injection mechanism 11, a liquid transfer mechanism 12, and two microorganism culture kettles connected in series, where the two microorganism culture kettles provided in this embodiment are a primary culture kettle 13 and a secondary culture kettle 14. The liquid injection mechanism 11 is used to inject a microbial culture solution into each microorganism culture kettle; the primary culture kettle 13 contains in situ sediment samples for microorganism enrichment; the liquid transfer mechanism 12 is used to transfer a microbial liquid from the primary culture kettle 13 to the secondary culture kettle 14.

The automatic sorting unit 2 includes a sorting operation kettle 21 and a plurality of pure culture kettles 22; and the sorting operation kettle 21 is connected to each pure culture kettle 22.

The sorting operation kettle 21 includes an internal three-axis moving mechanism 211 and at least one streaking pen tip box 212, where a plurality of streaking pen tips 213 are placed inside the streaking pen tip box 212, a moving Z-axis of the three-axis moving mechanism 211 is provided with a mechanical arm 214 capable of picking up the streaking pen tips 213 from the streaking pen tip box 212, and the mechanical arm 214 is used to drive the streaking pen tips 213 to perform sorting operations; and X-axis and Y-axis planes of the three-axis moving mechanism 211 are provided with a culture area composed of a plurality of petri dishes 215.

The secondary culture kettle 14 in the multi-level enrichment unit 1 is connected to the sorting operation kettle 21.

The pressure control unit 3 and the temperature control unit 4 are each connected to the central control unit 6, the sorting operation kettle 21, the primary culture kettle 13, the secondary culture kettle 14, and each pure culture kettle 22 respectively, and are used to control pressure and temperature within the sorting operation kettle 21, the primary culture kettle 13, the secondary culture kettle 14, and each pure culture kettle 22.

The data acquisition unit 5 is connected to the central control unit 6 and is used to acquire environmental data from the primary culture kettle 13 and the secondary culture kettle 14, as well as image data from the culture area inside the sorting operation kettle 21 in real time, and transmit the image data acquired to the central control unit 6 in real time.

The central control unit 6 is further connected to the primary culture kettle 13, the secondary culture kettle 14, the liquid injection mechanism 11, the liquid transfer mechanism 12, the mechanical arm 214, the pressure control unit 3, and the temperature control unit 4 respectively, where the central control unit 6 is used to control the online enrichment and automatic sorting apparatus for deep-sea microorganisms according to the data acquired by the data acquisition unit 5.

In the multi-level enrichment unit 1, the liquid injection mechanism 11 includes a continuous liquid injection pump 111 and two liquid injection valves 112, each liquid injection valve 112 is correspondingly provided on a microbial culture solution inlet pipeline of each microorganism culture kettle, and the continuous liquid injection pump 111 is connected to each liquid injection valve 112.

The liquid transfer mechanism 12 includes a sample transfer injection pump 121, a set of filters 122 and sample transfer valves 123, and a plunger pump 124; the sample transfer injection pump 121 is connected to the primary culture kettle 13 via a pressure-retaining valve 125, and the pressure-retaining valve 125 is used to retain constant pressure in the liquid transfer process; a set of filters 122 and sample transfer valves 123 are provided on a connecting pipeline between the primary culture kettle 13 and the secondary culture kettle 14, to achieve the transfer and subculturing of primary enrichment and secondary enrichment of the cultures; the plunger pump 124 is connected to the secondary culture kettle 14; and the plunger pump 124 is used to transfer the secondary culture kettle 14 to the sorting operation kettle 21.

The primary culture kettle 13 and the secondary culture kettle 14 have a same structure, with a magnetic stirrer 15 and a sampling valve 16 arranged at a bottom; the magnetic stirrer 15 is used to stir the culture solution inside the kettle; and the sampling valve 16 is used to periodically sample the culture for genetic sequencing analysis.

A top of the primary culture kettle 13 is provided with a sample transfer piston 131, and a middle part of microorganism culture kettle is provided with a protruding circular ring 132 with a narrower inner diameter, where the protruding circular ring 132 and the primary culture kettle 13 are of an integrated structure.

The sample transfer piston 131 is used to reduce a pressure difference required for transferring the microbial liquid to the secondary culture kettle 14; in this embodiment, an upper part of the sample transfer piston 131 is connected to the pressure-retaining valve 125 and the sample transfer injection pump 121, thereby forming a pressure-retaining layer at an upper part of the primary culture kettle 13; and the pressure-retaining layer is used to maintain the pressure required for primary enrichment of the culture and to facilitate the transfer and subculturing of the culture based on a smaller pressure difference. In addition, a pipeline is provided inside the sample transfer piston 131 and used to install the temperature sensor 521 and connect the gas injection valve 32, so as to facilitate suitable spatial arrangement of the interfaces.

The protruding circular ring 132 is used to prevent the sample transfer piston 131 from being excessively pressurized, which may damage the magnetic stirrer 15 at the bottom and the sensor interface in the data acquisition unit 5.

A bottom of the secondary culture kettle 14 is further provided with a sorting valve 141, the sorting valve 141 is connected to the plunger pump 124, and the plunger pump 124 is further connected to the microbial liquid reservoir 217 inside the sorting operation kettle 21; and the pipeline is used to transfer the enriched cultures under constant pressure to a next automatic sorting process.

In the automatic sorting unit 2, the sorting operation kettle 21 includes an upper kettle body and a lower kettle body that are hermetically connected; and the upper kettle body and the lower kettle body are connected and sealed via sealing gaskets and multiple screws 23 on an outer ring.

A pressure-resistant viewing window 216 is provided on an upper surface of the upper kettle body, and relatively large and made of pressure-resistant material, and is used to observe the interior of the sorting operation kettle 21.

A microbial liquid reservoir 217, the three-axis moving mechanism 211, at least one streaking pen tip box 212, the plurality of petri dishes 215, a pen tip recovery reservoir 218, and a sorting transfer port 219 are provided in the lower kettle body. A top view of the interior of the lower kettle body is as shown in FIG. 5.

The microbial liquid reservoir 217 is used to hold the to-be-sorted microbial liquid enriched by the secondary culture kettle 14.

The three-axis moving mechanism 211 is provided with three X-axis, Y-axis, and Z-axis sliding rails that vertically intersect. The Y-axis sliding rail 2112 is installed on the trajectory of the X-axis sliding rail 2111 via a connecting block, so as to enable the mechanical arm 214 to move laterally in a plane; the Z-axis sliding rail 2113 is installed on the Y-axis sliding rail 2112 via the connecting block, so as to enable the mechanical arm 214 to move vertically in the plane; the mechanical arm 214 is installed on the trajectory of the Z-axis sliding rail 2113 via the connecting block, so as to enable the mechanical arm 214 to move perpendicular to the plane.

The culture area is of a circular structure and includes four petri dishes 215, where a special culture medium for culturing specific individual colonies is pre-filled in each petri dish 215 to sort required special microbial species.

The pen tip recovery reservoir 218 is used to recycle used streaking pen tips 213.

The sorting transfer port 219 is connected to each pure culture kettle 22 via pipelines, ball valves 24, and quick-connect interfaces 25, where the sorting transfer port 219 is used to transfer the streaking pen tip 213 carrying individual colonies after sorting to the pure culture kettle 22.

The mechanical arm 214 includes: a vertical rod 2141, and a magnetic force sensor 2142 and a fixing block 2143 provided at both ends of the vertical rod 2141; the magnetic force sensor 2142 is respectively connected to the central control unit 6 and the fixing block 2143, the magnetic force sensor 2142 provides a magnetic force to the fixing block 2143, and the fixing block 2143 picks up the streaking pen tip 213 by the magnetic force.

The mechanical arm 214 is used to perform the following operations: picking up the streaking pen tip 213 from the streaking pen tip box 212; driving the streaking pen tip 213 to dip the microbial liquid in the microbial liquid reservoir 217 and the petri dishes 215; driving the streaking pen tip 213 to perform streaking and sorting in the petri dishes 215; and releasing the streaking pen tip 213 in the pen tip recovery reservoir 218 and the sorting transfer port 219.

The pure culture kettle 22 in this embodiment is used to provide multiple special microbial species for pure culture; before the experiment begins, a special culture solution needs to be injected into the pure culture kettle and the pure culture kettle is connected to the pressure control unit 3 for pressurization of the corresponding gas. The pure culture kettle 22 can be connected to the quick-connect interface 25 corresponding to the sorting transfer port 219 of the sorting operation kettle 21 to quickly transfer the sorted streaking pen tip 213 and the contained microorganism, thereby initiating pure culture using the pure culture kettle 22.

The pressure control unit 3 includes: a gas pressurization system 31, a plurality of gas injection valves 32, a plurality of PID control valves 33, and an exhaust valve 34.

The primary culture kettle 13, the secondary culture kettle 14, the sorting operation kettle 21, and each pure culture kettle 22 are respectively provided with one gas injection valve 32; and the gas pressurization system 31 is connected to each gas injection valve 32 via pipelines, connected to the central control unit 6 and used to perform gas pressurization for the primary culture kettle 13, the secondary culture kettle 14, the sorting operation kettle 21, and each pure culture kettle 22.

The primary culture kettle 13 and the secondary culture kettle 14 are further respectively provided with one PID control valve 33, and each PID control valve 33 is connected to the central control unit 6; a specified pressure threshold is set to the PID control valve 33 to establish a continuous reaction system in the primary culture kettle 13 and the secondary culture kettle 14, thereby achieving uniform mass transfer and continuous renewal of the culture medium, and enhancing the enrichment efficiency of the culture in the kettle.

The exhaust valve 34 is provided on the sorting operation kettle 21 and is used to exhaust gas from the sorting operation kettle 21.

The temperature control unit 4 includes: an air conditioning system 41.

The air conditioning system 41 is respectively connected to the central control unit 6, the primary culture kettle 13, the secondary culture kettle 14, the sorting operation kettle 21, and each pure culture kettle 22, and is used to control the temperature within the primary culture kettle 13, the secondary culture kettle 14, the sorting operation kettle 21, and each pure culture kettle 22.

The data acquisition unit 5 includes: a pressure sensing system 51, a temperature sensing system 52, a Raman measurement system 53, a dissolved oxygen measurement system 54, a pH measurement system 55, and an imaging system 56.

The pressure sensing system 51 and the temperature sensing system 52 are each connected to the central control unit 6, the primary culture kettle 13, the secondary culture kettle 14, the sorting operation kettle 21, and each pure culture kettle 22 respectively, and are respectively used to acquire the pressure and temperature inside the primary culture kettle 13, the secondary culture kettle 14, the sorting operation kettle 21, and each pure culture kettle 22 in real time and transmit the pressure and the temperature acquired to the central control unit 6 in real time; in this embodiment, the pressure sensing system 51 includes three pressure sensors 511, and the temperature sensing system 52 includes three temperature sensors 521, where one pressure sensor 511 and one temperature sensor 521 are respectively provided inside the primary culture kettle 13, the secondary culture kettle 14, and the sorting operation kettle 21.

The Raman measurement system 53, the dissolved oxygen measurement system 54, and the pH measurement system 55 are each connected to the central control unit 6, the primary culture kettle 13 respectively, and the secondary culture kettle 14, and are respectively used to perform real-time online observing of changes in chemical substance content, dissolved oxygen content, and pH value within each microorganism culture kettle, and transmit the changes in the chemical substance content, the dissolved oxygen content, and the pH value within each microorganism culture kettle that are observed to the central control unit 6 in real time.

In this embodiment, the Raman measurement system 53 includes: two sets of pressure-resistant observation chambers 531, two sets of observation valves 532, and two sets of exhaust valves 533, where the two sets of equipment are respectively provided inside the primary culture kettle 13 and the secondary culture kettle 14. The pressure-resistant observation chamber 531 is provided with a Raman measurement probe. At regular intervals, samples are taken via the observation valves 532 and transferred to the pressure-resistant observation chambers 531, where the Raman measurement probe is used to measure changes in chemical substance content and transmit the changes in chemical substance content measured to the central control unit 6 in real time; after the observation is completed, the samples in the pressure-resistant observation chamber 531 are emptied using the exhaust valve 533, waiting for next sampling.

The dissolved oxygen measurement system 54 and the pH measurement system 55 are respectively used to measure the dissolved oxygen content and pH value in the primary culture kettle 13 and the secondary culture kettle 14, so as to identify the progress of anaerobic oxidation reactions of microorganisms.

The imaging system 56 includes a camera 561 and an annular illumination lamp 562, where the camera 561 is provided above the pressure-resistant viewing window 216 at the top of the sorting operation kettle 21 and is connected to the central control unit 6. The camera 561 is used to acquire image data of the culture area inside the sorting operation kettle 21 in real time and transmit acquired image data to the central control unit 6 in real time. Additionally, the annular illumination lamp 562 is further provided inside the upper kettle body to facilitate observation of individual colonies and improve imaging quality.

The central control unit 6 includes: a computer host 61.

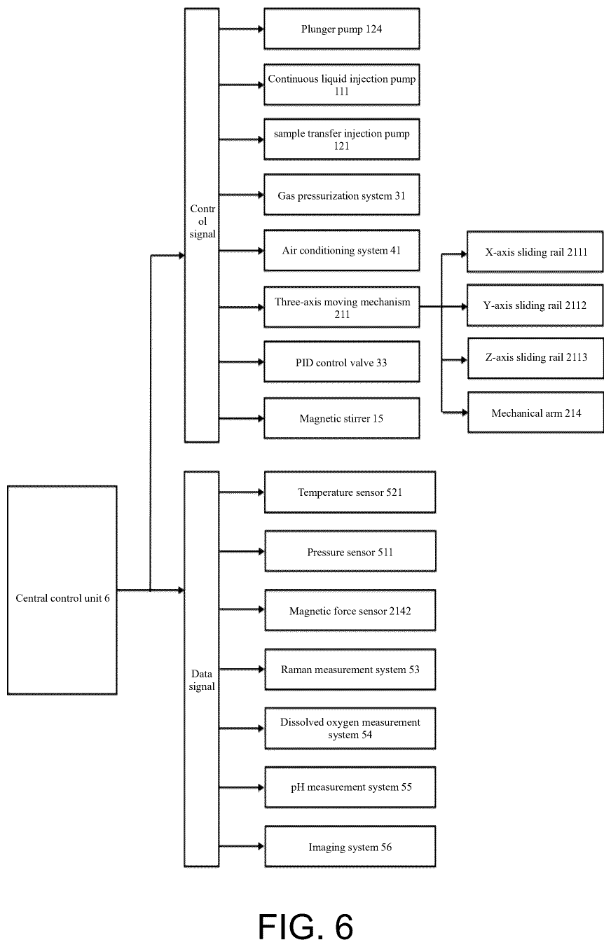

As shown in FIG. 6, the computer host 61 establishes in communication connection with the pressure sensing system 51, the temperature sensing system 52, the Raman measurement system 53, the dissolved oxygen measurement system 54, the pH measurement system 55, and the imaging system 56 in the data acquisition unit 5, and is used to receive the data acquired by the data acquisition unit 5.

The computer host 61 is further respectively connected to the liquid injection mechanism 11, the liquid transfer mechanism 12, the magnetic stirrers 15 in the primary culture kettle 13 and the secondary culture kettle 14, the mechanical arm 214, the pressure control unit 3, and the temperature control unit 4, and is used to control the online enrichment and automatic sorting apparatus for deep-sea microorganisms according to the data acquired by the data acquisition unit 5.

In a specific implementation process, prior to deep-sea microorganism enrichment, the primary culture kettle 13, the secondary culture kettle 14, the sorting operation kettle 21 and each pure culture kettle 22 are cleaned and sterilized, and pressure leak of the primary culture kettle 13, the secondary culture kettle 14, the sorting operation kettle 21 and each pure culture kettle 22 is tested; subsequently, each unit of the entire apparatus is installed, and all sensors and other electronic instruments within the apparatus are connected to the central control unit 6, and the instruments are activated, and connected to software of the computer host 61.

Subsequently, the liquid injection mechanism 11 is used to inject a pre-prepared microbial culture solution into each microorganism culture kettle at a specified flow rate; a special culture solution is pre-prepared for each petri dish 215 in the sorting operation kettle 21; the pre-prepared special culture solution is pre-injected into each pure culture kettle 22; the gas pressurization system 31 in the pressure control unit 3 is used, in combination with the pressure sensing system 51, to perform gas pressurization on each microorganism culture kettle, the sorting operation kettle 21 and each pure culture kettle 22 to simulate a deep-sea high-pressure environment; the PID control valve 33 is opened to set the pressure, forming a continuous environmental system; and the temperature control unit 4 is used, in combination with the temperature sensing system 52, to control the temperature of each microorganism culture kettle, the sorting operation kettle 21 and each pure culture kettle 22, maintaining the special temperature environment of the deep sea.

Sampled in situ sediment samples are transferred to the primary culture kettle 13, and the magnetic stirrer 15 is turned on, to initiate microorganism enrichment.

The Raman measurement system 53, the dissolved oxygen measurement system 54, and the pH measurement system 55 are used to respectively measure the changes in chemical substance content, dissolved oxygen content, and pH value in the primary culture kettle 14, and transmit the changes in chemical substance content, the dissolved oxygen content, and the pH value in the primary culture kettle 14 that are measured to the computer host 61 in the central control unit 6 in real time.

When the environmental data in the primary culture kettle 13 meets the first preset condition, the central control unit 6 controls the liquid transfer mechanism 12 to transfer the microbial liquid from the primary culture kettle 13 to the secondary culture kettle 14 for further enrichment culture; specifically, the sample transfer injection pump 121 is turned on, and the pressure-retaining valve 125 and the sample transfer valve 123 are opened, and pure water is injected to drive the sample transfer piston 131, such that the enriched microbial liquid enters the secondary culture kettle 14 for further culture.

When the environmental data in the secondary culture kettle 14 meets the second preset condition, the enrichment operation is completed, the plunger pump 124 is turned on and the sorting valve 141 is opened, and the microbial liquid in the secondary culture kettle 14 is transferred to the microbial liquid reservoir 217 in the sorting operation kettle 21.

Subsequently, the imaging system 56 is used to acquire image data from the culture area inside the sorting operation kettle 21 in real time and transmit the acquired image data to the central control unit 6 in real time.

The central control unit 6 controls the mechanical arm 214 to continuously picks up and releases the streaking pen tip 213, and drives the streaking pen tip 213 to perform streaking operations in each petri dish 215 in the culture area for sorting and culture for a preset duration.

The central control unit 6 drives the mechanical arm 214 to move by controlling the X-axis sliding rail 2111, the Y-axis sliding rail 2112, and the Z-axis sliding rail 2113. A specific process for each streaking operation is as follows:

The central control unit 6 controls the mechanical arm 214 to move to the streaking pen tip box 212, picks up the streaking pen tip 213, and subsequently controls the mechanical arm 214 to move to the petri dish 215 for streaking. After streaking, the mechanical arm 214 is controlled to depart from the culture area, move to a pen tip recovery reservoir 218, and release the used streaking pen tip 213.

After the microbial liquid has been cultured in the culture area for a period of time, when the central control unit 6 identifies the growth of multiple individual colonies in the petri dish 215 according to the image data of the culture area, the central control unit 6 controls the mechanical arm to transfer each individual colony to each pure culture kettle 22, and the sorting operation is completed.

Specifically, a transfer operation process is as follows:

The central control unit 6 controls the mechanical arm 214 to pick up the streaking pen tip 213, move to an individual colony for carrying, transfer the individual colony to a sorting transfer port 219 and release the streaking pen tip 213, the streaking pen tip 213 carrying the individual colony is transferred to the pure culture kettle 22; this step is repeated until all individual colonies are transferred to the pure culture kettle 22 for further pure culture and preservation.

The apparatus can perform continuous multi-level enrichment on cultures under high-pressure environments through dilution subculturing, and monitor environmental indicators of the culture system online. This achieves integration of enrichment and sorting apparatuses, transfer of the cultures under constant pressure and aseptic conditions, automatic streaking and sorting of the culture medium within the apparatus, and online monitoring of growth status of individual colonies, thereby improving the cultivability and acquisition efficiency of individual species of deep-sea microorganisms.

### Embodiment 5

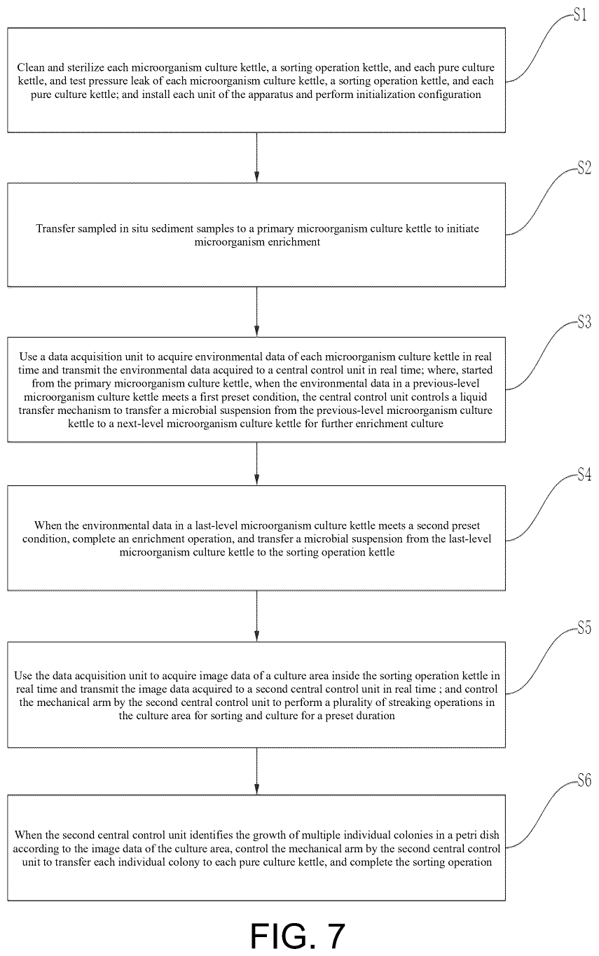

As shown in FIG. 7, this embodiment provides an online enrichment and automatic sorting method for deep-sea microorganisms under high-pressure environments based on the online enrichment and automatic sorting apparatus for deep-sea microorganisms under high-pressure environments described in Embodiment 4, and the method includes the following steps:
S1: cleaning and sterilizing each microorganism culture kettle, a sorting operation kettle, and each pure culture kettle, and testing pressure leak of each microorganism culture kettle, a sorting operation kettle, and each pure culture kettle; and installing each unit of the apparatus and performing initialization configuration;
S2: transferring sampled in situ sediment samples to a primary microorganism culture kettle to initiate microorganism enrichment;
S3: using a data acquisition unit to acquire environmental data of each microorganism culture kettle in real time and transmit the environmental data acquired to a first central control unit in real time; where,
started from the primary microorganism culture kettle, when the environmental data in a previous-level microorganism culture kettle meets a first preset condition, the central control unit controls a liquid transfer mechanism to transfer a microbial liquid from the previous-level microorganism culture kettle to a next-level microorganism culture kettle for further enrichment culture; and
S4: when the environmental data in a last-level microorganism culture kettle meets a second preset condition, completing an enrichment operation, and transferring a microbial liquid from the last-level microorganism culture kettle to the sorting operation kettle;
S5: using the data acquisition unit to acquire image data of a culture area inside the sorting operation kettle in real time and transmit the image data acquired to the central control unit in real time; and controlling the mechanical arm by the central control unit to perform a plurality of streaking operations in the culture area for sorting and culture for a preset duration;
S6: when the central control unit identifies the growth of multiple individual colonies in a petri dish according to the image data of the culture area, controlling the mechanical arm by the central control unit to transfer each individual colony to each pure culture kettle, and completing the sorting operation.

In step S1, the initialization configuration includes: using a liquid injection mechanism to inject a pre-prepared microbial culture solution into each microorganism culture kettle at a specified flow rate; pre-preparing a special culture medium for each petri dish in the sorting operation kettle, and pre-injecting the pre-prepared special culture solution into each pure culture kettle; using the pressure control unit to perform gas pressurization on each microorganism culture kettle □ the sorting operation kettle and each pure culture kettle to simulate a deep-sea high-pressure environment; and using the temperature control unit to control the temperature of each microorganism culture kettle □ the sorting operation kettle and each pure culture kettle to maintain the special deep-sea temperature environment.

In step S5, each streaking operation includes:
controlling, by the central control unit, the mechanical arm to move to a streaking pen tip box and to picks up the streaking pen tip, and subsequently controlling, by the second central control unit, the mechanical arm to move to the petri dish for streaking; after streaking, controlling, by the second central control unit, the mechanical arm to depart from the culture area, move to a pen tip recovery reservoir, and release the streaking pen tip.

In step S6, the controlling the mechanical arm by the central control unit to transfer each individual colony to each pure culture kettle includes:
controlling, by the central control unit, the mechanical arm to pick up the streaking pen tip, move to an individual colony for carrying, then be transferred to a sorting transfer port and release the streaking pen tip; and transferring the streaking pen tip carrying the individual colony to the pure culture kettle.

In a specific implementation process, prior to deep-sea microorganism enrichment, the primary culture kettle 13, the secondary culture kettle 14, the sorting operation kettle 21 and each pure culture kettle 22 are cleaned and sterilized, and pressure leak of the primary culture kettle 13, the secondary culture kettle 14, the sorting operation kettle 21 and each pure culture kettle 22 is tested; subsequently, each unit of the entire apparatus is installed, and all sensors and other electronic instruments within the apparatus are connected to the central control unit 6, and the instruments are activated, and connected to software of the computer host 61.

Subsequently, the liquid injection mechanism 11 is used to inject a pre-prepared microbial culture solution into each microorganism culture kettle at a specified flow rate; a special culture medium is pre-prepared for each petri dish 215 in the sorting operation kettle 21; the pre-prepared special culture solution is pre-injected into each pure culture kettle 22; the gas pressurization system 31 in the pressure control unit 3 is used, in combination with the pressure sensing system 51, to perform gas pressurization on each microorganism culture kettle, the sorting operation kettle 21 and each pure culture kettle 22 to simulate a deep-sea high-pressure environment; the PID control valve 33 is opened to set the pressure, forming a continuous environmental system; and the temperature control unit 4 is used, in combination with the temperature sensing system 52, to control the temperature of each microorganism culture kettle, the sorting operation kettle 21 and each pure culture kettle 22, maintaining the special temperature environment of the deep sea.

Sampled in situ sediment samples are transferred to the primary culture kettle 13, and the magnetic stirrer 15 is turned on, to initiate microorganism enrichment.

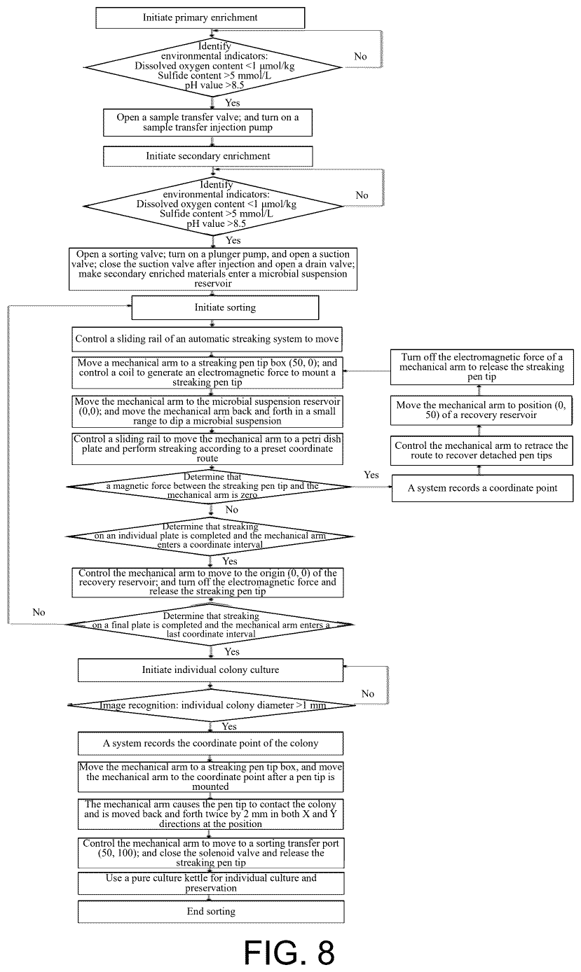

As shown in FIG. 8, after the primary culture kettle 13 is filled with enriched materials, primary enrichment is initiated. Environmental indicators are identified through the Raman measurement system 53, the dissolved oxygen measurement system 54, and the pH measurement system 55. If the environmental indicators are all met: dissolved oxygen content <1 μmol/kg, sulfide content >5 mmol/L (sulfide content obtained from the Raman measurement system 53), and pH value >8.5, sample transfer can be performed. The sample transfer valve 123 is opened and the sample transfer injection pump 121 is turned on, and the enriched materials are transferred to the secondary culture kettle 14 to initiate secondary enrichment. If any of these environmental indicators is not met, primary enrichment continues to be performed.

When secondary enrichment is initiated, environmental indicators are also identified through the Raman measurement system 53, the dissolved oxygen measurement system 54, and the pH measurement system 55. If the environmental indicators are all met: dissolved oxygen content <1 μmol/kg, sulfide content >5 mmol/L, and pH value >8.5, sorting can be performed. The sorting valve 141 is opened and the plunger pump 124 is turned on, and after a piston container thereof sucks 20 mL of the microbial liquid, the microbial liquid is injected and enters the microbial liquid reservoir 217 through the pipeline.

After the secondary enriched materials enter the microbial liquid reservoir 217, sorting is initiated. The central control unit 6 controls the X-axis sliding rail 2111, Y-axis sliding rail 2112, and Z-axis sliding rail 2113 of the three-axis moving mechanism 211 to drive the mechanical arm 214. A movement range of the three-axis sliding rail is (0, 0, 0) to (100, 100, 100), and the position of the mechanical arm 214 within the three-axis moving mechanism 211 is represented by coordinates within the movement range.

The mechanical arm 214 is then moved to position (50, 0)˜(80, 5) of the streaking pen tip box 212, and controls the coil of the magnetic force sensor 2142 in the mechanical arm 214 to be electrified to generate an electromagnetic force to pick up and mount the streaking pen tip 213. A total of 12 pen tips are mounted, with each pen tip spaced 5 coordinate points apart.

Next, the mechanical arm 214 is moved to position (0, 0) of the microbial liquid reservoir 217, and moved back and forth within a small range of (0, 0) to (5, 5) to dip the microbial liquid; the sliding rail is controlled, and the mechanical arm 214 is moved to four petri dishes 215 in the culture area for streaking according to preset coordinate routes, with each route having a coordinate system length of 20 and an interval length of 3. Each petri dish 215 can have 8 routes streaked. Thus, the streaking range for the first petri dish 215 is (20, 40˜60), (23, 40˜60) . . . (38, 40˜60), (41, 40˜60); the streaking range for the second petri dish 215 is (45, 60˜80), (48, 60˜80) . . . (63, 60˜80), (66, 60˜80); the streaking range for the third petri dish 215 is (45, 20˜40), (48, 20˜40) . . . (63, 20˜40), (66, 20˜40); and the streaking range for the fourth petri dish 215 is (70, 40˜60), (73, 40˜60) . . . (88, 40˜60), (91, 40˜60).

During the sorting and streaking process, the magnetic force sensor 2142 on the mechanical arm 214 can sense magnetic force. If the magnetic force is zero, it is determined that the streaking pen tip 213 has been detached, and the central control unit 6 records the coordinate point, controls the mechanical arm 214 to retrace the route at the coordinate point to recover the detached pen tip, the mechanical arm 214 is moved to position (0, 50) of the pen tip recovery reservoir 218, and the electromagnetic force of the mechanical arm 214 is adjusted to zero to release and discard the streaking pen tip 214; if the electromagnetic force is not zero, it is determined that the streaking pen tip 213 is functioning normally, and a streaking procedure continues to be performed until the mechanical arm 214 is identified as entering the coordinate intervals in each petri dish 215, namely at positions (41, 60), (66, 40), (66, 80), and (91, 60). In this case, the streaking operation for the petri dish 215 is completed. The mechanical arm 214 is moved to the pen tip recovery reservoir 218 at position (0, 50), and the magnetic force of the mechanical arm 214 is adjusted to zero to release and discard the streaking pen tip 213. Finally, the central control unit 6 determines whether the mechanical arm 214 has reached the last streaking coordinate position (91, 60) of the fourth petri dish 215. If the mechanical arm 214 has not reached the final position, the above streaking operation continues to be performed. If the mechanical arm 214 has reached the final position, the streaking operation ends, sorting is completed, and individual colony culture is initiated.

During individual colony culture, based on high-definition image acquisition by the imaging system 56 and the image recognition function of the central control unit 6, when an individual colony with a diameter >1 mm is identified, the central control unit 6 records the coordinate point of the colony, the mechanical arm 214 is moved to the streaking pen tip box 212 and then moved to the coordinate point after the streaking pen tip 213 is mounted, and the mechanical arm 214 causes the streaking pen tip 213 to contact the colony and is moved back and forth twice within a 2 mm range in the X and Y directions at the position. The mechanical arm 214 is then moved to position (50, 100) of the sorting transfer port 219, the electromagnetic force of the mechanical arm 214 is adjusted to zero, the ball valve 24 is opened, and the streaking pen tip 213 is released, allowing the streaking pen tip to fall into the connected pure culture kettle 22 for subsequent individual culture and preservation, thereby completing microorganism sorting.

In the method, continuous multi-level enrichment can be performed on cultures under high-pressure environments through dilution subculturing, and environmental indicators of the culture system are monitored online. This achieves integration of enrichment and sorting apparatuses, transfer of the cultures under constant pressure and aseptic conditions, automatic streaking and sorting of the culture medium within the apparatus, and online monitoring of growth status of individual colonies, thereby improving the cultivability and acquisition efficiency of individual species of deep-sea microorganisms.

Identical or similar reference numerals correspond to identical or similar components.

Terms describing positional relationships in the drawings are for exemplary illustration only and shall not be construed as limitations to this patent.

Obviously, the above embodiments of the present invention are merely examples to clearly illustrate the present invention and are not intended to limit the implementations of the present invention. For those of ordinary skill in the art, other variations or modifications in different forms can be made based on the above description. It is neither necessary nor possible to exhaustively list all implementations herein. Any modifications, equivalent substitutions, improvements, and the like made within the spirit and principles of the present invention shall be included within the scope of protection of the claims of the present invention.

What is claimed is:

1. An online enrichment apparatus for deep-sea microorganisms under high-pressure environments, comprising: a multi-level enrichment unit, a first pressure control unit, a first temperature control unit, a first data acquisition unit, and a first central control unit; wherein
the multi-level enrichment unit comprises a liquid injection mechanism, a liquid transfer mechanism, and at least two microorganism culture kettles connected in series; the liquid injection mechanism is used to inject a microbial culture solution into each microorganism culture kettle; a primary microorganism culture kettle contains in situ sediment samples for microorganism enrichment; the liquid transfer mechanism is used to transfer a microbial liquid from a previous-level microorganism culture kettle to a next-level microorganism culture kettle;
the first pressure control unit and the first temperature control unit are each connected to the first central control unit and each microorganism culture kettle respectively, and are respectively used to control pressure and temperature within each microorganism culture kettle;
the first data acquisition unit is connected to the first central control unit, and the first data acquisition unit is used to acquire environmental data from each microorganism culture kettle in real time and transmit the environmental data acquired to the first central control unit in real time;
the first data acquisition unit comprises: a pressure sensing system, a temperature sensing system, a Raman measurement system, a dissolved oxygen measurement system, and a pH measurement system;
the pressure sensing system and the temperature sensing system are each connected to the first central control unit and each microorganism culture kettle respectively, and are respectively used to acquire the pressure and temperature within each microorganism culture kettle in real time and transmit the pressure and temperature acquired to the first central control unit in real time;
the Raman measurement system, the dissolved oxygen measurement system, and the pH measurement system are each connected to the first central control unit and each microorganism culture kettle respectively, and are respectively used to perform real-time online observing of changes in chemical substance content, dissolved oxygen content, and pH value within each microorganism culture kettle, and transmit the changes in the chemical substance content, the dissolved oxygen content, and the pH value within each microorganism culture kettle that are observed to the first central control unit in real time; and
the first central control unit is further connected to each microorganism culture kettle, the liquid injection mechanism, the liquid transfer mechanism, the first pressure control unit, and the first temperature control unit respectively; and the first central control unit is used to control the online enrichment apparatus for deep-sea microorganisms under high-pressure environments according to the data acquired by the first data acquisition unit.

2. The online enrichment apparatus for deep-sea microorganisms under high-pressure environments according to claim 1, wherein, in the multi-level enrichment unit, the liquid injection mechanism comprises a continuous liquid injection pump and a plurality of liquid injection valves, each liquid injection valve is correspondingly provided on a microbial culture solution inlet pipeline of each microorganism culture kettle, and the continuous liquid injection pump is connected to each liquid injection valve;
the liquid transfer mechanism includes a sample transfer injection pump, multiple sets of filters and sample transfer valves, as well as a plunger pump; the sample transfer injection pump is respectively connected to all microorganism culture kettles except for a last-level microorganism culture kettle, and one set of filters and the sample transfer valve is provided on a connection pipeline between each two microorganism culture kettles connected in series; and the plunger pump is connected to the last-level microorganism culture kettle;
each microorganism culture kettle has a same structure, with a magnetic stirrer and a sampling valve provided at a bottom; and
tops of all the microorganism culture kettles except for a last-level microorganism culture kettle are each provided with a sample transfer piston, and middle parts of all the microorganism culture kettles are each provided with a protruding circular ring; the sample transfer piston is used to reduce a pressure difference required for transferring the microbial liquid from the previous-level microorganism culture kettle to the next-level microorganism culture kettle; and the protruding circular ring is used to prevent the sample transfer piston from being excessively pressurized.

3. The online enrichment apparatus for deep-sea microorganisms under high-pressure environments according to claim 1, wherein the first pressure control unit comprises: a gas pressurization system, a plurality of gas injection valves, and a plurality of PID control valves;
each microorganism culture kettle is provided with one gas injection valve; the gas pressurization system is connected to each gas injection valve via pipelines, and the gas pressurization system is connected to the first central control unit, and is used to perform gas pressurization on each microorganism culture kettle;
each microorganism culture kettle is further provided with one PID control valve, and each PID control valve is connected to the central control unit and is used to control formation of a continuous pressure environment within the microorganism culture kettles connected in series;
the first temperature control unit comprises: an air conditioning system; and
the air conditioning system is respectively connected to the first central control unit and each microorganism culture kettle, and is used to control the temperature within each microorganism culture kettle.

4. An online enrichment and automatic sorting apparatus for deep-sea microorganisms under high-pressure environments, comprising an enrichment apparatus and a sorting apparatus connected in sequence, wherein the enrichment apparatus is specifically the online enrichment apparatus for deep-sea microorganisms under high-pressure environments according to claim 1; the sorting apparatus comprises: an automatic sorting unit, a second pressure control unit, a second temperature control unit, a second data acquisition unit, and a second central control unit;
the automatic sorting unit comprises a sorting operation kettle and a plurality of pure culture kettles; the sorting operation kettle is connected to each pure culture kettle;
the sorting operation kettle comprises an internal three-axis moving mechanism and at least one streaking pen tip box, wherein a plurality of streaking pen tips are placed inside the streaking pen tip box, a moving Z-axis of the three-axis moving mechanism is provided with a mechanical arm capable of picking up the streaking pen tips from the streaking pen tip box, and the mechanical arm is used to drive the streaking pen tips to perform sorting operations; X-axis and Y-axis planes of the three-axis moving mechanism are provided with a culture area composed of a plurality of petri dishes;
the sorting operation kettle comprises an upper kettle body and a lower kettle body that are hermetically connected;
a pressure-resistant viewing window is provided on an upper surface of the upper kettle body for observing an interior of the sorting operation kettle;
a microbial liquid reservoir, the three-axis moving mechanism, at least one streaking pen tip box, the plurality of petri dishes, a pen tip recovery reservoir, and a sorting transfer port are provided in the lower kettle body;
the microbial liquid reservoir is used to hold a to-be-sorted microbial liquid;
the three-axis moving mechanism is provided with three X-axis, Y-axis, and Z-axis sliding rails that vertically intersect, wherein the mechanical arm is provided on the Z-axis sliding rail;
the mechanical arm comprises: a vertical rod, and a magnetic force sensor and a fixing block provided at both ends of the vertical rod; the magnetic force sensor is respectively connected to the second central control unit and the fixing block, the magnetic force sensor provides a magnetic force to the fixing block, and the fixing block picks up the streaking pen tip by the magnetic force;
the mechanical arm is used to perform the following operations: picking up the streaking pen tip from the streaking pen tip box and installing the streaking pen tip; driving the streaking pen tip to dip the microbial liquid in the microbial liquid reservoir and the petri dishes; driving the streaking pen tip to perform streaking and sorting in the petri dishes; and releasing the streaking pen tip in the pen tip recovery reservoir and the sorting transfer port;
a culture medium for cultivating individual colonies is pre-filled in the petri dishes to sort required microbial species;
the pen tip recovery reservoir is used to collect used streaking pen tips;
the sorting transfer port is connected to each pure culture kettle via pipelines and quick-connect interfaces, wherein the sorting transfer port is used to transfer the streaking pen tip carrying individual colonies after sorting to the pure culture kettle;
the second pressure control unit and the second temperature control unit are each connected to the second central control unit, the sorting operation kettle, and each pure culture kettle respectively, and are respectively used to control pressure and temperature within the sorting operation kettle, and each pure culture kettle;
the second data acquisition unit is connected to the second central control unit and is used to acquire image data of the culture area inside the sorting operation kettle in real time and transmit the image data acquired to the second central control unit in real time; and
the second central control unit is further connected to the mechanical arm, the second pressure control unit, and the second temperature control unit respectively, wherein the second central control unit is used to control the online automatic sorting apparatus for deep-sea microorganisms under high-pressure environments according to the image data acquired by the second data acquisition unit.

5. The online enrichment and automatic sorting apparatus for deep-sea microorganisms under high-pressure environments according to claim 4, wherein the second pressure control unit comprises: a gas pressurization system, an exhaust valve, and a plurality of gas injection valves;
the sorting operation kettle and each pure culture kettle are respectively provided with one gas injection valve; the gas pressurization system is connected to each gas injection valve via pipelines, and the gas pressurization system is connected to the second central control unit and used to perform gas pressurization for the sorting operation kettle and each pure culture kettle;
the exhaust valve is provided on the sorting operation kettle and is used to exhaust gas from the sorting operation kettle;
the second temperature control unit comprises: an air conditioning system;
the air conditioning system is respectively connected to the second central control unit, the sorting operation kettle, and each pure culture kettle, and is used to control the temperature within the sorting operation kettle, and each pure culture kettle;
the second data acquisition unit comprises a pressure sensing system, a temperature sensing system, and an imaging system;
the pressure sensing system and the temperature sensing system are each connected to the second central control unit, the sorting operation kettle, and each pure culture kettle respectively, and are used to acquire the pressure and temperature within the sorting operation kettle, and each pure culture kettle in real time and transmit the pressure and temperature acquired to the second central control unit in real time; and
the imaging system is provided at a top of the sorting operation kettle and is connected to the second central control unit, wherein the imaging system is used to acquire image data of the culture area inside the sorting operation kettle in real time and transmit the image data acquired to the second central control unit in real time.

6. The online enrichment and automatic sorting apparatus for deep-sea microorganisms under high-pressure environments according to claim 4, wherein a last-level microorganism culture kettle in the multi-level enrichment unit is connected to the microbial liquid reservoir inside the sorting operation kettle; and
the microbial liquid reservoir is used to hold the to-be-sorted microbial liquid enriched by the last-level microorganism culture kettle.

7. An online enrichment and automatic sorting method for deep-sea microorganisms under high-pressure environments based on the online enrichment and automatic sorting apparatus for deep-sea microorganisms under high-pressure environments according to claim 4, comprising the following steps:
S1: cleaning and sterilizing each microorganism culture kettle, a sorting operation kettle, and each pure culture kettle, and testing pressure leak of each microorganism culture kettle, the sorting operation kettle, and each pure culture kettle; and installing each unit of the apparatus and performing initialization configuration;
S2: transferring sampled in situ sediment samples to a primary microorganism culture kettle to initiate microorganism enrichment;
S3: using a first data acquisition unit to acquire environmental data of each microorganism culture kettle in real time and transmit the environmental data acquired to a first central control unit in real time; wherein,
started from the primary microorganism culture kettle, when the environmental data in a previous-level microorganism culture kettle meets a first preset condition, the first central control unit controls a liquid transfer mechanism to transfer a microbial liquid from the previous-level microorganism culture kettle to a next-level microorganism culture kettle for further enrichment culture;
S4: when the environmental data in a last-level microorganism culture kettle meets a second preset condition, completing an enrichment operation, and transferring a microbial liquid from the last-level microorganism culture kettle to the sorting operation kettle;
S5: using a second data acquisition unit to acquire image data of a culture area inside the sorting operation kettle in real time and transmit the image data acquired to a second central control unit in real time; and controlling the mechanical arm by the second central control unit to perform a plurality of streaking operations in the culture area for sorting and culture for a preset duration; and
S6: when the second central control unit identifies the growth of multiple individual colonies in a petri dish from the image data of the culture area, controlling the mechanical arm by the second central control unit to transfer each individual colony to each pure culture kettle, and completing the sorting operation.

8. The online enrichment and automatic sorting method for deep-sea microorganisms under high-pressure environments according to claim 7, wherein in step S1, the initialization configuration comprises: using a liquid injection mechanism to inject a pre-prepared microbial culture solution into each microorganism culture kettle at a specified flow rate; pre-preparing a culture medium for each petri dish in the sorting operation kettle, and pre-injecting the pre-prepared culture solution into each pure culture kettle; using a first pressure control unit and a second pressure control unit to perform gas pressurization on each microorganism culture kettle, the sorting operation kettle, and each pure culture kettle to simulate a deep-sea high-pressure environment; and using a first temperature control unit and a second temperature control unit to control the temperature of each microorganism culture kettle, the sorting operation kettle, and each pure culture kettle to maintain the deep-sea temperature environment;
in step S5, each streaking operation comprises:
controlling, by the second central control unit, the mechanical arm to move to a streaking pen tip box and to picks up the streaking pen tip, and subsequently controlling, by the second central control unit, the mechanical arm to move to the petri dish for streaking; after streaking, controlling, by the second central control unit, the mechanical arm to depart from the culture area, move to a pen tip recovery reservoir, and release the streaking pen tip;
in step S6, the controlling the mechanical arm by the second central control unit to transfer each individual colony to each pure culture kettle includes:
controlling, by the second central control unit, the mechanical arm to pick up the streaking pen tip, move to an individual colony for carrying, then be transferred to a sorting transfer port and release the streaking pen tip; and transferring the streaking pen tip carrying the individual colony to the pure culture kettle.
